(19) United States
(12) Patent Application Publication
(10) Pub. No.: US 2024/0373810 A1
(43) Pub. Date: Nov. 14, 2024

(54) SOYBEAN OLIGOSACCHARIDE-RELATED KOMPETITIVE ALLELE-SPECIFIC PCR (KASP) MARKER AND USE THEREOF

(71) Applicant: JIANGSU ACADEMY OF AGRICULTURAL SCIENCES, Nanjing, Jiangsu (CN)

(72) Inventors: Huatao CHEN, Nanjing, Jiangsu (CN); Yanzhe LI, Nanjing, Jiangsu (CN); Wei ZHANG, Nanjing, Jiangsu (CN); Hongmei ZHANG, Nanjing, Jiangsu (CN); Wenjing XU, Nanjing, Jiangsu (CN); Xiaoyan CUI, Nanjing, Jiangsu (CN); Xiaoqing LIU, Nanjing, Jiangsu (CN); Qiong WANG, Nanjing, Jiangsu (CN); Xin CHEN, Nanjing, Jiangsu (CN)

(21) Appl. No.: 18/248,954

(22) PCT Filed: Nov. 15, 2022
(86) PCT No.: PCT/CN2022/131907

§ 371 Date: Apr. 13, 2023

(30) Foreign Application Priority Data
Sep. 16, 2022 (CN) 202211126441.7

(51) Int. Cl. A01H 6/54; A01H 5/10

(52) U.S. Cl. CPC A01H 6/542; A01H 5/10

(57) ABSTRACT

Provided is a soybean oligosaccharide-related kompetitive allele-specific PCR (KASP) marker and use thereof, belonging to the technical field of molecular breeding. The KASP marker includes one or two of S18_51868868 T/G and S10_38081012 T/C, where the S18_51868868 T/G is a base T or a base G at a 51868868bp position on chromosome 18 of a soybean genome; and the S10_38081012 T/C is a base T or a base C at a 38081012bp position of chromosome 10 of the soybean genome. In the present disclosure, the KASP marker can accurately genotype the traits of raffinose and stachyose contents. Through genotyping, it is found that a soybean germplasm with a genotype GG has a higher raffinose content than that of a soybean germplasm with a genotype TT, and a soybean germplasm with a genotype TT has a higher stachyose content than that of a soybean germplasm with a genotype CC.

### CROSS REFERENCE TO RELATED APPLICATION

The present application is a national stage application of International Patent Application No. PCT/CN2022/131907, filed on Nov. 15, 2022, which claims priority of the Chinese Patent Application No. 202211126441.7, filed with the China National Intellectual Property Administration (CNIPA) on Friday, Sep. 16, 2022, and entitled “SOYBEAN OLIGOSACCHARIDE-RELATED KOMPETITIVE ALLELE-SPECIFIC PCR (KASP) MARKER AND USE THEREOF”, both of which are incorporated herein by reference in their entireties.

### REFERENCE TO SEQUENCE LISTING

A computer readable XML file entitled “HLPCTP20230201024_seqlist.xml”, that was created on Mar. 13, 2023, with a file size of about 7,337 bytes, contains the sequence listing for this application, has been filed with this application, and is hereby incorporated by reference in its entirety.

### TECHNICAL FIELD

The present disclosure belongs to the technical field of molecular breeding, and in particular relates to a soybean oligosaccharide-related kompetitive allele-specific PCR (KASP) marker and use thereof.

### BACKGROUND

Soybean oligosaccharides are the general term of soluble saccharides in soybean grains, mainly including sucrose, raffinose, and stachyose. Studies have indicated that oligosaccharides have desirable physical and chemical properties, and show safety, non-toxicity, low sweetness, and strong thermal stability. Moreover, the oligosaccharides have extremely important physiological functions, such as diarrhea prevention, anti-tumor effects, and liver protection. Therefore, soy bean oligosaccharides, as a kind of health food, are widely used in foods such as beverages, yogurt, aquatic products, jams, pastries, and bread, and have a broad market potential.

Soybean oligosaccharides play an important role in human health and plant growth and development. However, there is poor utilization of soy bean resources currently, and there are few special soybean varieties. Cultivation of the special soybean varieties has a desirable development prospect. Soybean oligosaccharides are typical quantitative traits controlled by multiple genes, and are easily affected by external environmental factors such as soil environment and climate change. In conventional screening methods, the oligosaccharides of each soybean germplasm are determined, which has long identification period, high cost, and time-consuming and laborious deficiencies. Accordingly, the conventional methods cannot meet the demands of stable and efficient screening.

### SUMMARY

In view of this, an objective of the present disclosure is to provide a soybean oligosaccharide-related KASP marker and use thereof.

To achieve the above objective, the present disclosure provides the following technical solutions:

The present disclosure provides a soybean oligosaccharide-related KASP marker, including one or two of S18_51868868 T/G and S10_38081012 T/C, where the S18_51868868 T/G is a base T or a base G at a 51868868bp position on chromosome 18 of a soy bean genome; and the S10_38081012 T/C is a base T or a base C at a 38081012bp position of chromosome 10 of the soybean genome.

In some embodiments, a soybean with the base T at the S18_51868868 T/G has a low raffinose content; and a soybean with the base G at the S18_51868868 T/G has a high raffinose content.

In some embodiments, a soybean with the base T at the S10_38081012 T/C has a high stachyose content; and a soybean with the base C at the S10_38081012 T/C has a low stachyose content.

The present disclosure further provides a primer pair for detecting the KASP marker, including one or two of an S18_51868868 T/G primer pair and an S10_38081012 T/C primer pair; where

the S18_51868868 T/G primer pair has an upstream primer F1 with a sequence of SEQ ID NO: 1, an upstream primer F2 with a sequence of SEQ ID NO: 2, and a downstream primer R with a sequence of SEQ ID NO: 3; and

the S10_38081012 T/C primer pair has an upstream primer F1 with a sequence of SEQ ID NO: 4, an upstream primer F2 with a sequence of SEQ ID NO: 5, and a downstream primer R with a sequence of SEQ ID NO: 6.

The present disclosure further provides a kit for detecting the KASP marker, including the primer pair above.

The present disclosure further provides use of the KASP marker, the primer pair, or the kit in germplasm breeding of soy bean with improved oligosaccharide.

The present disclosure provides a method for screening a soybean with a high oligosaccharide content, including the following steps:

conducting PCR amplification using a genomic DNA of a soybean to be tested as a template with the primer pair, followed by genotyping according to a fluorescence signal of a PCR amplification product.

In some embodiments, if a genotype at a 51868868bp position of the chromosome 18 is GG and/or if a genotype at a 38081012bp position of the chromosome 10 is TT in a genome of a soy bean to be tested, the soy bean to be tested has a high oligosaccharide content.

Compared with the prior art, the embodiments of the present disclosure has the following beneficial effects:

The present disclosure provides a soy bean oligosaccharide-related KASP marker. In the present disclosure, single nucleotide polymorphism (SNP) sites significantly associated with oligosaccharides are analyzed by genome-wide association study (GWAS), and two KASP markers are designed. The accuracy rate for detecting a high raffinose content by using S18_51868868 T/G can be as high as 91.3%, and the accuracy rate for detecting a high stachyose content by using the S10_38081012 T/C can be as high as 93.5%. As a result, the KASP marker may accurately genotype the traits of raffinose and stachyose contents. Through genotyping, it is found that a soy bean with a genotype GG has a higher raffinose content than that of a soybean with a genotype TT, and a soybean with a genotype TT has a higher stachyose content than that of a soybean with a genotype CC. Therefore, the KASP marker can be used for the breeding of soybean germplasms with a high oligosaccharide content. Moreover, the KASP marker is of great significance in the selection of early breeding, reducing the workload of soybean breeding, and accelerating the process of soy bean breeding.

### BRIEF DESCRIPTION OF THE DRAWINGS

FIGS. 1A-1C show resequencing results of natural populations, where FIG. 1A is a phylogenetic tree; FIG. 1B is the analysis of principal components; and FIG. 1C is the analysis of linkage disequilibrium;

FIGS. 2A-2F show Manhattan plots and Q-Q plots of the GWAS analysis of soybean germplasm oligosaccharides in two environments in 2020 and 2021, where FIG. 2A, FIG. 2B, and FIG. 2C are the GWAS results of stachyose, raffinose, and sucrose in 2020, respectively; and FIG. 2D, FIG. 2E, and FIG. 2F are the GWAS results of stachyose, raffinose, and sucrose in 2021, respectively; and

FIGS. 3A-3B show genotyping of the KASP marker on different soy bean germplasms, where FIG. 3A is a genotyping diagram of raffinose; and FIG. 3B is a genotyping diagram of stachyose.

### DETAILED DESCRIPTION OF THE EMBODIMENTS

The present disclosure will be further described below with reference to the examples and accompanying drawings.

The present disclosure provides a soybean oligosaccharide-related KASP marker, including one or two of S18_51868868 T/G and S10_38081012 T/C, where the S18_51868868 T/G is a base T or a base G at a 51868868bp position on chromosome 18 of a soybean genome; and the S10_38081012 T/C is a base T or a base C at a 38081012bp position of chromosome 10 of the soy bean genome.

In the present disclosure, the KASP marker includes one or two of S18_51868868 T/G and S10_38081012 T/C, both of which are related to the soybean oligosaccharide content. As a preferred embodiment, a soy bean oligosaccharide-related KASP marker is provided, where the KASP marker is S18_51868868 T/G, and the S18_51868868 T/G is a base T or a base G at a 51868868bp position on chromosome 18 of a soybean genome. As another preferred embodiment, a soybean oligosaccharide-related KASP marker is provided, where the KASP marker is S10_38081012 T/C, and the S10_38081012 T/C is a base T or a base C at a 38081012bp position on chromosome 10 of the soybean genome. As yet another preferred embodiment, a soybean oligosaccharide-related KASP marker is provided, where the KASP marker includes S18_51868868 T/G and S10_38081012 T/C, the S18_51868868 T/G is a base T or a base G at a 51868868bp position on chromosome 18 of a soybean genome; and the S10_38081012 T/C is a base T or a base C at a 38081012bp position of chromosome 10 of the soy bean genome.

In the present disclosure, a soybean with the base T at the S18_51868868 T/G has preferably a low raffinose content; and a soy bean with the base G at the S18_51868868 T/G has preferably a high raffinose content. A soybean with the base T at the S10_38081012 T/C has preferably a high stachyose content; and a soy bean with the base C at the S10_38081012 T/C has preferably a low stachyose content.

The present disclosure further provides a primer pair for detecting the KASP marker, including one or two of an S18_51868868 T/G primer pair and an S10_38081012 T/C primer pair; where

the S18_51868868 T/G primer pair has an upstream primer F1 with a sequence of SEQ ID NO: 1, an upstream primer F2 with a sequence of SEQ ID NO: 2, and a downstream primer R with a sequence of SEQ ID NO: 3; and

the S10_38081012 T/C primer pair has an upstream primer FI with a sequence of SEQ ID NO: 4, an upstream primer F2 with a sequence of SEQ ID NO: 5, and a downstream primer R with a sequence of SEQ ID NO: 6.

In the present disclosure, a primer pair of the KASP marker includes one or two of an S18_51868868 T/G primer pair and an S10_38081012 T/C primer pair. As a preferred embodiment, a primer pair for detecting the KASP marker is further provided, where the primer pair of the KASP marker is an S18_51868868 T/G primer pair. The S18_51868868 T/G has an upstream primer F1 with a sequence of SEQ ID NO: 1, an upstream primer F2 with a sequence of SEQ ID NO: 2, and a downstream primer R with a sequence of SEQ ID NO: 3. As a preferred embodiment, a primer pair for detecting the KASP marker is further provided, where the primer pair of the KASP marker is an S10_38081012 T/C primer pair. The S10_38081012 T/C has an upstream primer F1 with a sequence of SEQ ID NO: 4, an upstream primer F2 with a sequence of SEQ ID NO: 5, and a downstream primer R with a sequence of SEQ ID NO: 6. As yet another preferred embodiment, a primer pair for detecting the KASP marker is further provided, where the primer pair of the KASP marker includes an S18_51868868 T/G primer pair and an S10_38081012 T/C primer pair. The S18_51868868 T/G has an upstream primer F1 with a sequence of SEQ ID NO: 1, an upstream primer F2 with a sequence of SEQ ID NO: 2, and a downstream primer R with a sequence of SEQ ID NO: 3. The S10_38081012 T/C has an upstream primer F1 with a sequence of SEQ ID NO: 4, an upstream primer F2 with a sequence of SEQ ID NO: 5, and a downstream primer R with a sequence of SEQ ID NO: 6.

The present disclosure further provides a kit for detecting the KASP marker, including the primer pair.

In the present disclosure, the kit includes preferably a PCR amplification reaction solution. More preferably, the PCR amplification reaction solution includes one or two of the S18_51868868 T/G primer pair and the S10_38081012 T/C primer pair. The PCR amplification reaction solution further includes preferably a soybean sample DNA template and 2×KASP Master mix.

The present disclosure further provides use of the KASP marker, the primer pair, or the kit in germplasm breeding of soy bean with improved oligosaccharide.

The present disclosure provides a method for screening a soybean with a high oligosaccharide content, including the following steps:

conducting PCR amplification with the primer pair using a genomic DNA of a soybean to be tested as a template, followed by genotyping according to a fluorescence signal of a PCR amplification product.

In the present disclosure, if a genotype at a 51868868bp position of chromosome 18 is GG and/or if a genotype at a 38081012bp position of chromosome 10 is TT in a genome of a soy bean to be tested, the soy bean to be tested has a high oligosaccharide content. If the genotype at the 51868868bp position of chromosome 18 is TT and/or if the genotype at the 38081012bp position of chromosome 10 is CC in the genome of the soybean to be tested, the soybean to be tested has a low oligosaccharide content.

In the present disclosure, at the 51868868bp position of chromosome 18 in the soybean genome, if the genotype is GG, a soybean to be tested has a high raffinose content; if the genotype is TT, the soybean to be tested has a low raffinose content. The raffinose content of a soybean germplasm with genotype GG is higher than that of a soybean germplasm with genotype TT, with an average increase of 18.87% to 26.20%. At the 38081012bp position of chromosome 10 in the soybean genome, if the genotype is TT, a soybean to be tested has a high stachyose content; if the genotype is CC, the soybean to be tested has a low stachyose content. The stachyose content of a soybean germplasm with genotype TT is higher than that of a soy bean germplasm with genotype CC, with an average increase of 21.89% to 24.05%. In the present disclosure, if a genotype at a 51868868bp position of chromosome 18 is GG and if a genotype at a 38081012bp position of chromosome 10 is TT in a genome of a soybean to be tested, the soybean to be tested has a significantly-increased oligosaccharide content.

In the present disclosure, PCR amplification is conducted with the primer pair using a genomic DNA of a soybean to be tested as a template. A PCR amplification system includes preferably: 5 μL of a 20 ng/μL to 30 ng/μL soybean sample DNA template. 5 μL of 2×KASP Master mix, and 0.14 μL of KASP Assay Mix (F1: F2: R=1:1:1). The PCR amplification includes: initial denaturation at 94° C. for 15 min; denaturation at 94° C. for 20 sec, and annealing at 61° C. to 55° C. for 60 sec with a total of 10 cycles (each cycle being decreased by 0.6° C.); and denaturation at 94° C. for 20 sec, and annealing at 55° C. for 60 sec, with a total of 26 cycles.

The technical solutions provided by the present disclosure will be described in detail below with reference to examples, but the examples should not be construed as limiting the claimed scope of the present disclosure.

### Example 1

(1) Associated groups: 264 representative soybeans from all over China were selected, including 52 local species and 212 cultivated species, and 19 more wild soy beans were collected. These samples constituted the micro-core germplasm resources, in a total of 283 parts. The soy bean materials were mainly selected from the Huang-Huai-Hai region and the ecoregion in South China, and the rest came from the ecoregion in North China.

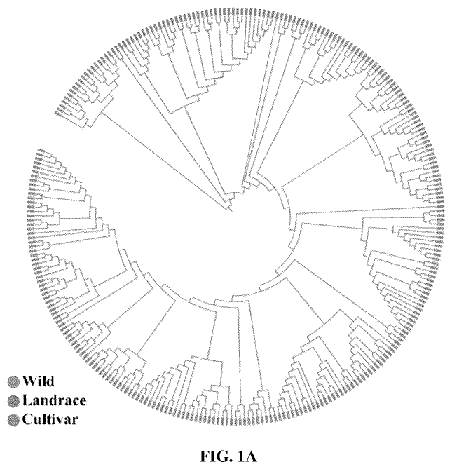

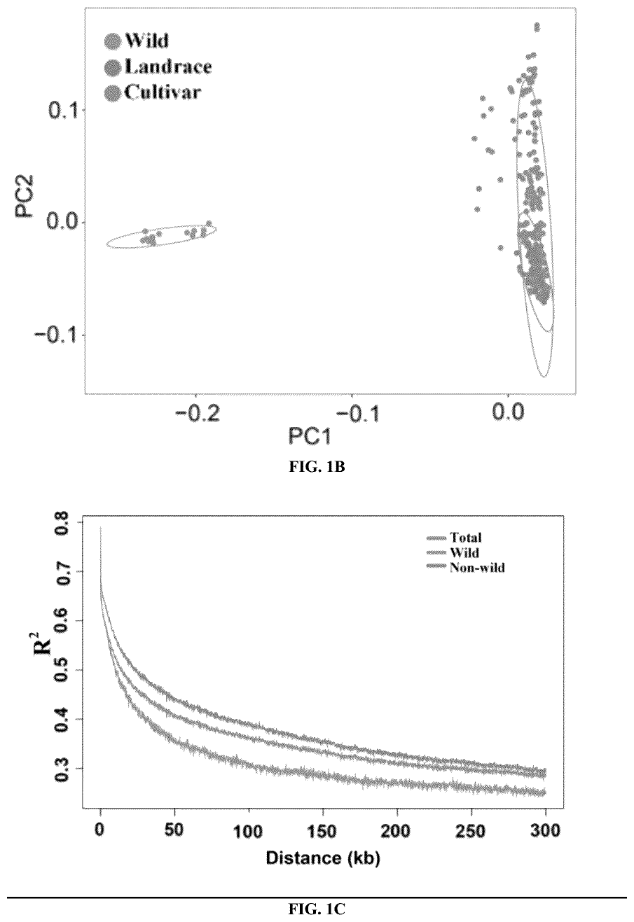

(2) Resequencing: the 283 materials were resequenced, with an average sequencing depth of 12.4×, and 10,210,329 SNP markers were obtained. A soybean phylogenetic tree was constructed based on 3,319,306 high-quality SNPs, which could effectively differentiate wild soy beans, landraces, and improved varieties, where the landraces and improved varieties were evolved and artificially selected from the wild soybeans (FIG. 1A). Principal component analysis (FIG. 1B) shows that the wild soybeans are effectively distinguished from the landraces and improved varieties. The landraces and improved varieties are closer, and there is an obvious difference between the wild and non-wild soybeans. Linkage disequilibrium (LD) analysis (FIG. 1C) showed that all materials had an LD value of approximately 106 kb, where the wild soybean had an LD value of approximately 33 kb, and the non-wild soybean had an LD value of approximately 120 kb.

(3) GWAS analysis of oligosaccharides: the SNP markers used for natural population GWAS came from resequencing, and the high-density physical map included a total of 2,597,425 SNPs. The GWAS was calculated using a Genome Associated Prediction Integrated Tool (GAPIT) algorithm package based on R software, and the GWAS was conducted using a mixed linear model (MLM) to control false-positive association sites. With P≤1/2597425=3.85×10⁻⁷, −LogP≥6.4 as a significant threshold, when an SNP site had a threshold of −LogP≥6.4, it was considered as a significant association site; when the SNP site had a threshold of 5≤−LogP<6.4, it was considered as a potential association site.

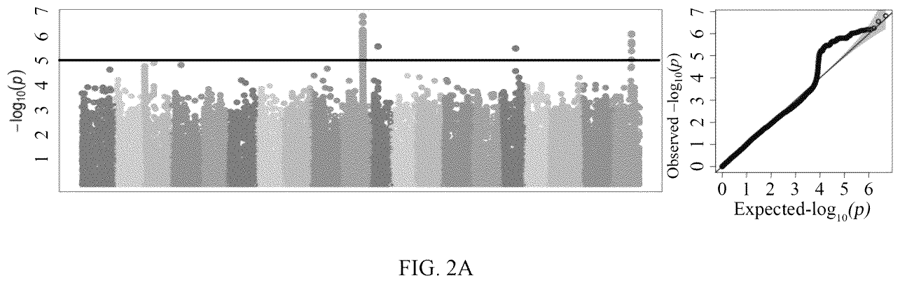

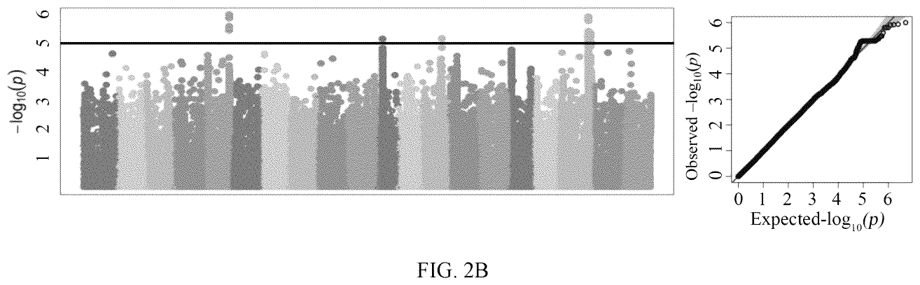

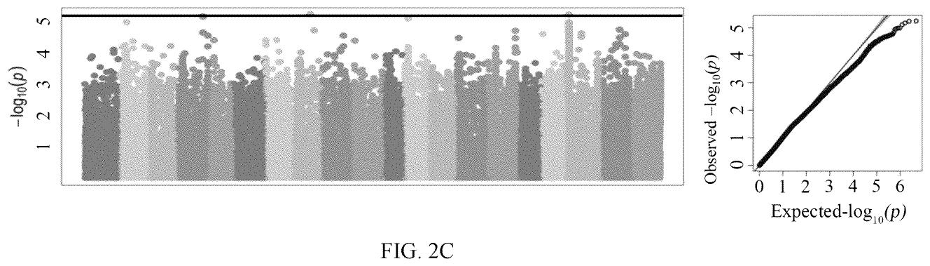

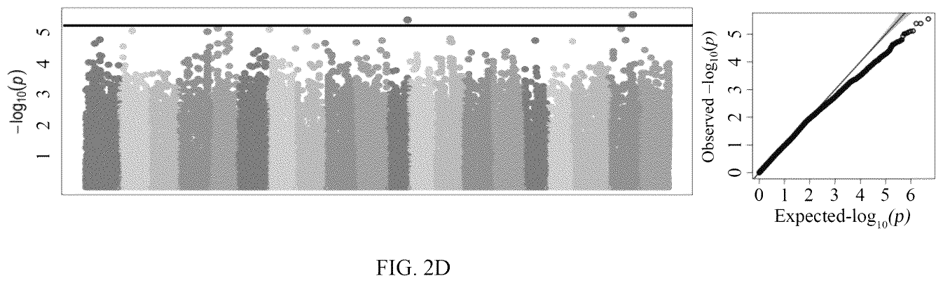

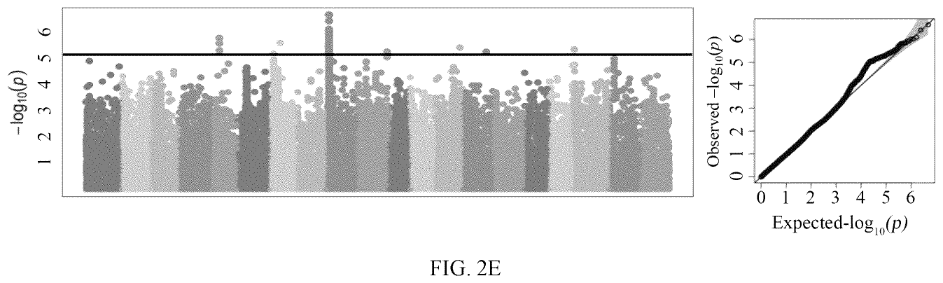

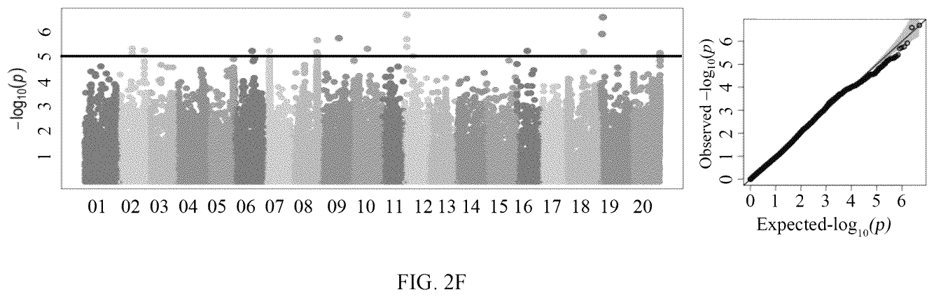

The oligosaccharide contents of soybean natural populations harvested in Hainan Base in 2020 and Nanjing Liuhe Base in 2021 were separately detected by high-performance liquid chromatography (HPLC), and GWAS was conducted on phenotypic data of the oligosaccharide contents in the two years (FIGS. 2A-2F).

TABLE 1
Number of SNP sites associated with soybean
oligosaccharides in natural populations

| Oligosaccharide component | Year | Location | −log10 (P) ≥ 5 |
|---|---|---|---|
| Sucrose | 2020 | Sanya | 4 |
| | 2021 | Nanjing | 25 |
| Raffinose | 2020 | Sanya | 83 |
| | 2021 | Nanjing | 224 |
| Stachyose | 2020 | Sanya | 526 |
| | 2021 | Nanjing | 9 |

As shown in Table 1, a total of 871 associated SNPs were obtained in the two years.

As shown in FIGS. 2A-2F, a total of 613 SNP sites were associated in 2020. There were 83 SNPs associated with the raffinose, including 5 SNPs on chromosome 5, 1 SNP on chromosome 11, 2 SNPs on chromosome 13, and 75 SNPs on chromosome 18. A single SNP site had a phenotypic variation interpretation rate of 6.56% to 8.10%. There were 526 SNPs associated with the stachyose, including 502 SNPs on chromosome 10, 1 SNP on each of chromosome 11, 14, and 16, and 21 SNPs on chromosome 20. A single SNP site had a phenotypic variation interpretation rate of 8.74% to 12.53%. There were 4 SNPs associated with the sucrose, which were located on chromosomes 4, 8, 12, and 18, respectively. A single SNP site had a phenotypic variation interpretation rate of 7.73% to 7.98%.

A total of 258 SNPs were associated in 2021. 224 SNPs associated with the raffinose were detected, including 3 SNPs on chromosome 5, 2 SNPs on chromosome 7, a maximum of 211 SNPs on chromosome 9, and 5 SNPs on chromosome 10. A single SNP site had a phenotypic variation interpretation rate of 20.47% to 21.18%. There were 9 SNPs associated with the stachyose, most of which were distributed on chromosomes 11 and 19. A single SNP site had a phenotypic variation interpretation rate of 98.13%. There were 25 SNPs associated with the sucrose, most of which were distributed on chromosomes 2, 8, 12, and 20. A single SNP site had a phenotypic variation interpretation rate of 62.19% to 63.13%. The SNPs significantly correlated with each oligosaccharide component were shown in Table 2.

Some significant SNP sites could be detected in the two years' environment. SNPs associated with the raffinose were co-localized on chromosomes 5, 13, and 18, and SNPs associated with the stachyose were co-localized on chromosome 11.

TABLE 2
SNPs significantly correlated with each oligosaccharide component

| | | | | | | Highly- | Maximum |
|---|---|---|---|---|---|---|---|
| | | Oligosac- | | Number | | significant | of −log10(P) |
| | | charide | Chromo- | of | | SNP | value |
| Year | Location | component | some | SNPs | Interval position | position | (−log10P max) |
| 2020 | Sanya | Raffinose | 5 | 5 | 40306495-40406880 | 40347558 | 5.99 |
| | | Raffinose | 18 | 75 | 51385538-55705745 | 52012350 | 5.93 |
| | | Stachyose | 10 | 502 | 37975495-38142527 | 37983091 | 6.81 |
| | | Stachyose | 20 | 21 | 33869071-3869420 | 33869418 | 6.11 |
| 2021 | Nanjing | Raffinose | 9 | 211 | 5202601-5385811 | 5224523 | 6.64 |
| | | Raffinose | 10 | 5 | 49321321-9321340 | 49321337 | 5.24 |
| | | Stachyose | 11 | 2 | 31904885-1904888 | 31904885 | 5.38 |
| | | Stachyose | 19 | 2 | 20563162-9525205 | 39525205 | 5.55 |
| | | Sucrose | 8 | 5 | 40939542-1851207 | 41780063 | 5.67 |
| | | Sucrose | 12 | 4 | 4957877-15564483 | 4957890 | 5.72 |

### Example 2

A soybean oligosaccharide-related KASP marker was provided, where the KASP marker included significantly-associated SNP sites S18_51868868 (T/G) and S10_38081012 (T/C) obtained in Example 1.

Three primers were designed, including an upstream primer F1, an upstream primer F2, and a downstream primer R. The F1 and F2 included FAM and VIC fluorescent linker sequences (underlined), respectively, and their sequences are shown in Table 3:

TABLE 3
Specific primers for KASP marker

| Primer name | Primer sequence |
|---|---|
| S18_51868868 F1 | GAAGGTGACCAAGTTCATGCTTCTTTCTGATTGTTTAGTCT (SEQ ID NO: 1) |
| S18_51868868 F2 | GAAGGTCGGAGTCAACGGATTTCTTTCTGATTGTTTAGTCG (SEQ ID NO: 2) |
| S18_51868868 R | AACCTGTACCCAGACAACACA (SEQ ID NO: 3) |
| S10_38081012 F1 | GAAGGTGACCAAGTTCATGCTAATCTTGACGGGGAAAACAT (SEQ ID NO: 4) |
| S10_38081012 F2 | GAAGGTCGGAGTCAACGGATTAATCTTGACGGGGAAAACAC (SEQ ID NO: 5) |
| S10_38081012 R | GGCACCAAAACATGGGGAAC (SEQ ID NO: 6) |

### Example 3

A soybean oligosaccharide-related KASP marker was provided, where the KASP marker was a significantly-associated SNP site S18_51868868 (T/G) obtained in Example 1. Three primers were designed, including an upstream primer F1, an upstream primer F2, and a downstream primer R, with sequences of SEQ ID NO: 1, SEQ ID NO: 2, and SEQ ID NO: 3, respectively.

Example 4

A soybean oligosaccharide-related KASP marker was provided, where the KASP marker was a significantly-associated SNP site S10_38081012 (T/C) obtained in Example 1.

Three primers were designed, including an upstream primer F1, an upstream primer F2, and a downstream primer R, with sequences of SEQ ID NO: 4, SEQ ID NO: 5, and SEQ ID NO: 6, respectively.

### Example 5

A kit of a KASP marker of S18_51868868 (T/G) was provided, where the kit included an upstream primer F1, an upstream primer F2, and a downstream primer R of the S18_51868868 (T/G), with sequences of SEQ ID NO: 1, SEQ ID NO: 2, and SEQ ID NO: 3, respectively.

### Example 6

A kit of a KASP marker of S10_38081012 (T/C) was provided, where the kit included an upstream primer F1, an upstream primer F2, and a downstream primer R of the S10_38081012 (T/C), with sequences of SEQ ID NO: 4, SEQ ID NO: 5, and SEQ ID NO: 6, respectively.

### Example 7

A kit of a KASP marker of S18_51868868 (T/G) and S10_38081012 (T/C) was provided, where the kit included upstream primers F1, upstream primers F2, and downstream primers R of the S18_51868868 (T/G) and the S10_38081012 (T/C), with sequences of SEQ ID NO: 1 to SEQ ID NO: 6.

### Example 8

A method for screening a soybean with a high oligosaccharide content included the following steps:

(1) Extraction of DNA of a soybean to be tested: young soybean leaves and small steel beads were added into a 2 mL centrifuge tube, and then quickly put into liquid nitrogen for storage. The centrifuge tube was put on a plant tissue grinder for grounding (30 Hz, 40 sec), and 600 μL of preheated CTAB was added for quickly mixing, and shaken from time to time in a 65° C. water bath for 30 min. An equal amount of a 24:1 mixture of chloroform: isoamyl alcohol was added, inverted gently, centrifuged at 12,000 rounds per minute (r) for 5 min, and a resulting supernatant was collected to conduct a secondary extraction. 100 μL of 5M NaCl solution and an equal volume of isopropanol (pre-cooled) were added, shaken gently, and an obtained mixture was placed in a refrigerator for 30 min. The mixture was centrifuged at 12,000 r for 5 min, waste liquid was discarded, and 600 μL of 70% ethanol was added to pop a resulting precipitate. The centrifugation was repeated once, 20 μL of ddH₂O was added, and a final DNA sample was stored in a refrigerator.

(2) A KASP marker was developed for the SNP sites S18_51868868 (T/G) and S10_38081012 (T/C) that were significantly associated with soybean oligosaccharides. The marker had sequences shown in Table 3. PCR amplification was conducted with corresponding primers F1, F2, and R using the DNA sample of the soy bean genome extracted in step (1) as a template in a QuantStudio5 real-time fluorescent quantitative PCR instrument to obtain PCR amplification products. The PCR amplification system included: 5 μL of the soybean sample DNA template (25 ng/μL), 5 μL of 2×KASP Master mix, and 0.14 μL of KASP Assay Mix (F1: F2: R=1:1:1). The reaction conditions of PCR amplification are shown in Table 4.

(3) After the reaction was completed, the fluorescence data of an obtained reaction product was read on the QuantStudio5 real-time fluorescent quantitative PCR instrument. 40 soybean samples were amplified and genotyped on a real-time fluorescent quantitative PCR instrument using the primer pairs of KASP molecular marker in Table 3.

TABLE 4
PCR amplification conditions

| | | | | Number of cycles | |
|---|---|---|---|---|---|
| Step | Process | Temperature | Time | in each step | |
| 1 | Initial | 94° C. | 15 min | 1 | cycle |
| | denaturation | | | | |
| 2 | Denaturation | 94° C. | 20 sec | 10 | cycles |
| | Annealing | 61° C.-55° C. | 60 sec (each | | |
| | | | cycle decreased | | |
| | | | by 0.6° C.) | | |
| 3 | Denaturation | 94° C. | 20 sec | 26 | cycles |
| | Annealing | 55° C. | 60 sec | | |

TABLE 5
Oligosaccharide contents of soybean
germplasms with different genotypes

| Oligosaccharide | | | Mean of content | |
|---|---|---|---|---|
| component | Marked SNP | Genotype | 2020 | 2021 |
| Stachyose | S10_38081012 | TT | 27.06 | 30.12 |
| | | CC | 21.83 | 24.71 |
| Raffinose | S18_51868868 | GG | 10.37 | 14.37 |
| | | TT | 7.29 | 10.11 |

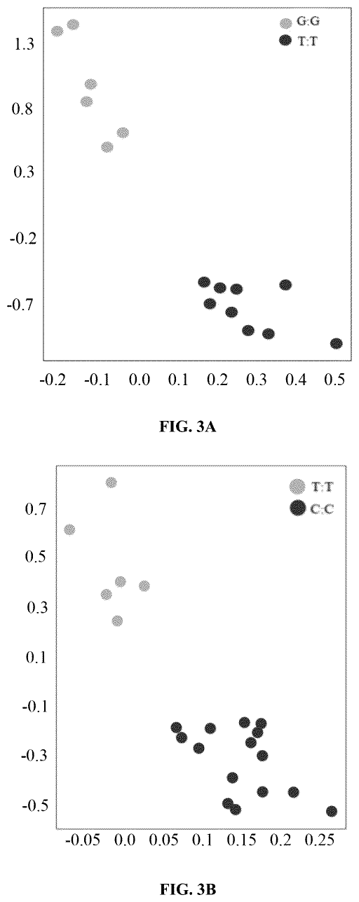

The results in FIGS. 3A-3B and Table 5 showed that: The molecular marker primer for S18_51868868 (T/G) could clearly separate the two genotypes. The blue dots near the Y axis represented soybean germplasms with a high raffinose content and carrying a G allele variation site, while the red dots near the X axis represented soybean germplasms with a low raffinose content and carrying a T allele variation site (FIG. 3A). The raffinose content of soybean germplasm with a genotype GG was generally higher than that of soybean germplasm with a genotype TT, showing an average increase of 18.87% to 26.20%. The molecular marker primer for S10_38081012 (T/C) could clearly separate the two genotypes. The blue dots near the Y axis represented soybean germplasms with a high stachyose content and carrying a T allele variation site, while the red dots near the X axis represented soybean germplasms with a low stachyose content and carrying a C allele variation site (FIG. 3B). The stachyose content of soybean germplasm with a genotype TT was generally higher than that of soybean germplasm with a genotype CC, showing an average increase of 21.89% to 24.05%. The accuracy rate for detecting the high raffinose content by using S18_51868868 T/G could be as high as 91.3%, and the accuracy rate for detecting the high stachyose content by using S10_38081012 T/C could be as high as 93.5%. The two markers both had an ideal effect in detecting the content of soy bean oligosaccharides.

In the present disclosure, through two different environmental tests in Sanya and Nanjing regions, the oligosaccharide content of soybean populations under different environmental conditions is measured, and the GWAS analysis is conducted. This makes the SNP sites of oligosaccharides obtained by association analysis more reliable, and makes the discovery of oligosaccharide-related candidate genes and the development of genetic markers more accurate. 2 KASP markers are designed and developed by the present disclosure. Through genotyping, it is found that the raffinose content of soy bean germplasm with a genotype GG is higher than that of soy bean germplasm with a genotype TT; the stachyose content of soybean germplasm with a genotype TT is higher than that of soybean germplasm with a genotype CC. The two KASP molecular markers can be used in the breeding of soybean oligosaccharide germplasms. Therefore, the KASP molecular marker of the present disclosure can accurately genotype the traits of raffinose and stachyose contents. The molecular marker may also be applied to early large-scale germplasm screening, and assist the molecular breeding of soy beans functionally.

The above description of examples is merely provided to help understand the method of the present disclosure and a core idea thereof. It should be noted that several improvements and modifications may be made by those of ordinary skill in the art without departing from the principle of the present disclosure, and these improvements and modifications should also fall within the protection scope of the present disclosure. Various amendments to these embodiments are apparent to those of professional skill in the art, and the general principles defined herein may be implemented in other embodiments without departing from the spirit or scope of the present disclosure. Thus, the present disclosure is not limited to the examples shown herein but falls within the widest scope consistent with the principles and novel features disclosed herein.

1. A soybean oligosaccharide-related kompetitive allele-specific PCR (KASP) marker, comprising one or two of S18_51868868 T/G and S10 38081012 T/C, wherein the S18_51868868 T/G is a base T or a base G at a 51868868bp position on chromosome 18 of a soybean genome; and the S10_38081012 T/C is a base T or a base C at a 38081012bp position of chromosome 10 of the soybean genome.

2. The KASP marker according to claim 1, wherein a soybean with the base T at the S18_51868868 T/G has a low raffinose content; and a soybean with the base G at the S18_51868868 T/G has a high raffinose content.

3. The KASP marker according to claim 1, wherein a soybean with the base T at the S10_38081012 T/C has a high stachyose content; and a soybean with the base C at the S10_38081012 T/C has a low stachyose content.

4. A primer pair for detecting the KASP marker according to claim 1, comprising one or two of an S18_51868868 T/G primer pair and an S10_38081012 T/C primer pair; wherein
the S18_51868868 T/G primer pair has an upstream primer F1 with a sequence of SEQ ID NO: 1, an upstream primer F2 with a sequence of SEQ ID NO: 2, and a downstream primer R with a sequence of SEQ ID NO: 3; and
the S10_38081012 T/C primer pair has an upstream primer F1 with a sequence of SEQ ID NO: 4, an upstream primer F2 with a sequence of SEQ ID NO: 5, and a downstream primer R with a sequence of SEQ ID NO: 6.

5. A kit for detecting the KASP marker according to claim 1, comprising one or two of an S18_51868868 T/G primer pair and an S10_38081012 T/C primer pair; wherein
the S18_51868868 T/G primer pair has an upstream primer F1 with a sequence of SEQ ID NO: 1, an upstream primer F2 with a sequence of SEQ ID NO: 2, and a downstream primer R with a sequence of SEQ ID NO: 3; and
the S10_38081012 T/C primer pair has an upstream primer F1 with a sequence of SEQ ID NO: 4, an upstream primer F2 with a sequence of SEQ ID NO: 5, and a downstream primer R with a sequence of SEQ ID NO: 6.

6. (canceled)

7. A method for screening a soybean with a high oligosaccharide content, comprising the following steps:
conducting PCR amplification using a genomic DNA of a soybean to be tested as a template with the primer pair according to claim 4, followed by genotyping according to a fluorescence signal of a PCR amplification product.

8. The method according to claim 7, wherein if a genotype at a 51868868bp position of the chromosome 18 is GG and/or if a genotype at a 38081012bp position of the chromosome 10 is TT in a genome of the soybean to be tested, the soybean to be tested has a high oligosaccharide content.


SEQUENCE LISTING (6 sequences)

SEQ ID NO: 1 - DNA, 41 bp, synthetic construct
  source 1..41: mol_type = other DNA; note = S18_51868868 F1
gaaggtgaccaagttcatgcttctttctgattgtttagtct

SEQ ID NO: 2 - DNA, 41 bp, synthetic construct
  source 1..41: mol_type = other DNA; note = S18_51868868 F2
gaaggtcggagtcaacggatttctttctgattgtttagtcg

SEQ ID NO: 3 - DNA, 21 bp, synthetic construct
  source 1..21: mol_type = other DNA; note = S18_51868868 R
aacctgtacccagacaacaca

SEQ ID NO: 4 - DNA, 41 bp, synthetic construct
  source 1..41: mol_type = other DNA; note = S10_38081012 F1
gaaggtgaccaagttcatgctaatcttgacggggaaaacat

SEQ ID NO: 5 - DNA, 41 bp, synthetic construct
  source 1..41: mol_type = other DNA; note = S10_38081012 F2
gaaggtcggagtcaacggattaatcttgacggggaaaacac

SEQ ID NO: 6 - DNA, 20 bp, synthetic construct
  source 1..20: mol_type = other DNA; note = S10_38081012 R
ggcaccaaaacatggggaac